(19) United States
(12) Patent Application Publication
(10) Pub. No.: US 2026/0242708 A1
(43) Pub. Date: Aug. 20, 2026

(54) TOROIDAL BIOREACTORS AND USES THEREOF

(71) Applicant: Toro Bio, Inc., Redwood City, CA (US)

(72) Inventor: Umberto SCARFOGLIERO, Redwood City, CA (US)

(73) Assignee: Toro Bio, Inc., Redwood City, CA (US)

(21) Appl. No.: 19/641,039

(22) Filed: Apr. 7, 2026

Related U.S. Application Data
(63) Continuation of application No. PCT/US2024/051242, filed on Oct. 14, 2024.
(60) Provisional application No. 63/590,440, filed on Oct. 15, 2023.

(51) Int. Cl. C12M 1/12; C12M 1/00; C12M 1/04; C12M 1/34; C12M 1/42; C12M 3/00; C12M 3/04; F16K 31/44

(52) U.S. Cl. CPC C12M 23/06; C12M 23/22; C12M 23/24; C12M 23/48; C12M 27/10; C12M 29/04; C12M 29/20; C12M 35/02; C12M 35/06; C12M 41/00; C12M 41/12; C12M 41/26; C12M 41/34; C12M 41/36; C12M 47/06; F16K 31/44

(57) ABSTRACT

Provided herein are v-shaped (or generally v-shaped) toroidal bioreactors that can be rotated about an axis. The shape of the container, as well as the ability to rotate it, gives the system the additional degrees of freedom needed for handling several operations commonly performed on cells, including growing, expanding, activating, transducing and manipulating cells in a closed system.

### CROSS-REFERENCE TO RELATED APPLICATIONS

This application is a continuation of International App. No. PCT/US2024/051242 filed Oct. 14, 2024, which claims priority to and the benefit of U.S. Prov. App. No. 63/590,440 filed Oct. 15, 2023, each of which is hereby incorporated by reference in its entirety.

### FIELD

The present disclosure relates generally to bioreactors, and more specifically to bioreactors and components thereof used for growing, expanding, activating, transducing and manipulating cells in a closed system.

### BACKGROUND

Current methods of cell expansions use rigid flasks, bioreactor containers with membranes or bioreactor bags, and different operations often require extracting fluid from these containers, for sampling for example, because the fluid volume grew beyond what the container can hold. These containers are usually housed in an incubator or device which may control temperature, humidity, CO2 levels, pressure and other environmental parameters, control fluids in and out and in some cases provide degrees of motion for the cells.

The bottlenecks of the approaches currently adopted in the state of the art are similar, albeit to various degrees. They take significant space, affecting not only the need for clean-room space, but also making logistics of moving consumables challenging. They are not designed to be automated, but rather designed to be used by skilled scientists or operators. They also require constant sampling for obtaining actual cell count and viability, the main analytics parameters for cell growth, exposing the cell culture to risks such as contamination and human errors. Rigid flasks also require several manual transfers, usually opening the container, which create bottlenecks in the handling. Such flasks need to be operated in cleanrooms or laminar flow biosafety cabinets and create contamination risks.

Common bioreactors are bulky, complex to install with multiple tubings, each with their own peculiarities, requiring complex training and operation controls, making their use prone to quality errors.

In addition, the recent promises of allogeneic therapies, as well as iPSC, require the handling of fluid volumes usually handled via pipettes in open fashion, as well as much larger volumes during the same process. This poses challenges for the scalability of the process since the product cannot be produced in a closed system.

Thus, what is needed in the art are improved bioreactor system design that can more efficiently and effectively grow, expand, activate, transduce and manipulate cells in a closed system.

### BRIEF SUMMARY

In some aspects, provided herein are bioreactor containers and systems incorporating such bioreactor containers that are suitable for use in growing cells and tissue culture applications. The systems herein may also be generalized for use in different particles, from beads to small molecules to viruses, either in biological, chemical, pharmaceutical or other applications.

The systems described herein provide a v-shaped (or generally v-shaped) toroidal bioreactor that can be rotated along its axis. The shape of the container, as well as the ability to rotate it, gives the system the additional degrees of freedom needed for handling several operations commonly performed on cells, such as enrichment, activation, transduction, expansion, and “fill and finish”. The ability to spin the bioreactor allows automatic cell resuspension in a closed system, and provides centrifugal force to be used for the desired manipulation of cells, beads or particles. The V-shape cross-section allows the air-fluid surface to increase as the volume increases, and it provides the ability to position the fluid, and the particles in it, along the outer surface of the container as it spins. The location as a function of time is going to be based on the fluid and particles properties, such as mass and viscosity for example. This in turn enables the system to obtain imaging of cells or particles without opening the system or extracting a sample. The presence of micro cavities on the top of the outer surface allows the selection of small amounts of fluids that would otherwise require open system pipetting. These cavities are filled with fluid during the rotation, but that can be extracted as the rotation is stopped. The proposed system has a compact form factor that can be easily handled by automation systems.

In one aspect, provided is a bioreactor comprising:
a toroidal container having a cross-sectional profile comprising a top plate, a first side-wall, and a second side-wall,
at least one fluid inlet configured to provide fluid communication between a stationary external fluid source and an interior of the toroidal container as the toroidal bioreactor rotates about its axis;
at least one fluid outlet configured to provide fluid communication between the interior of the toroidal container and a stationary exterior fluid reservoir as the toroidal bioreactor rotates about its axis; and
at least one micro cavity feature integrated with a top edge of the first side-wall and configured to sample an aliquot of fluid contained within the toroidal container.

In another aspect, provided is a system comprising:
at least one toroidal container comprising:
   a cross-sectional profile comprising a top plate, a first side-wall, and a second side-wall, and
   at least one micro cavity feature integrated with a top edge of the first side-wall and configured to sample an aliquot of fluid contained within the toroidal container; and
at least one drive mechanism comprising:
   a frame for mounting the toroidal container, wherein the frame is configured to allow the toroidal container to spin around its rotational axis; and
   at least one drive motor;
wherein the drive mechanism is configured to control the angular velocity of the toroidal container as a function of time.

In another aspect, provided is method for culturing cells using any of the bioreactors described herein. In some embodiments, the method comprises:
introducing cells into a growth medium contained within a bioreactor comprising:
   a toroidal container having a cross-sectional profile comprising a top plate, a first side-wall, and a second side-wall, and
   at least one micro cavity feature integrated with a top edge of the first side-wall and configured to:
   (i) sample an aliquot of fluid contained within the toroidal container when a force is applied to a valve mechanism integrated with the at least one micro cavity feature; and
   (ii) deliver the fluid aliquot to a stationary external fluid sample container;
aerating and/or agitating the growth medium contained within the toroidal container by rotating the toroidal container about its axis at a specified angular velocity; and
monitoring cell growth within the growth medium.

In some embodiments, cell growth within the growth medium is monitored by performing high speed imaging of cells suspended in the growth medium. In other embodiments, cell growth within the growth medium is monitored by performing high speed imaging of cells attached to an interior surface of the first side wall of the toroidal container. In another embodiment, cell growth within the growth medium is monitored by performing high speed imaging of the cells and fluids contained in the micro-cavities designed for imaging, such as being made by optically transparent materials, such as polycarbonate (PC). In other embodiments, cell growth within the growth medium is monitored by sampling an aliquot of the growth medium using the at least one micro cavity feature by increasing an angular velocity for rotation of the toroidal container so that it exceeds a specified minimum angular velocity threshold, and delivering the aliquot of the growth medium to a stationary external sample container for testing. In other embodiments, a combination of any of the aforementioned methods to monitor cell growth within the growth medium may be employed.

### DESCRIPTION OF THE FIGURES

The present application can be understood by reference to the following description taken in conjunction with the accompanying figures.

FIG. 1 depicts an exemplary toroidal bioreactor 10 housed in the frame 20, according to one embodiment of the disclosed devices and systems.

FIG. 2A depicts a simplified cross-sectional view of an exemplary toroidal bioreactor container 100, according to one embodiment of the disclosed devices and systems.

FIG. 2B depicts a cross-sectional view of an exemplary toroidal bioreactor container 100, according to one embodiment of the disclosed devices and systems.

FIG. 3 depicts a top angle view of an exemplary toroidal bioreactor 10 housed in the frame 20.

FIG. 4 shows a cross-section section of an exemplary toroidal bioreactor 10 including the frame 20, with fluid 30 inside the bioreactor container 100.

FIG. 5 depicts a side angle view of an exemplary bioreactor container 100 positioned within the drive mechanism 400 is equipped with a magnetic system 440.

FIGS. 6A-6D depict various cross-sectional views of an exemplary bioreactor container 100 with specialized sections of cavity 160 for the selection of various quantities of fluids, like the ones otherwise handled by pipetting.

FIG. 7 depicts the main components of exemplary drive mechanism 400.

FIG. 8 depicts an exemplary method for using the toroidal bioreactors described herein.

FIG. 9 provides a non-limiting schematic illustration of a computing device or system in accordance with one or more examples of the present disclosure.

FIG. 10 provides a non-limiting example of the cross-section of a toroidal bioreactor container have an S-shaped outer side-wall.

FIG. 11 provides a non-limiting example of the side view (upper) and top view (lower) of a toroidal bioreactor container.

### DETAILED DESCRIPTION

The following description sets forth exemplary systems, methods, parameters and the like. It should be recognized, however, that such description is not intended as a limitation on the scope of the present disclosure but is instead provided as a description of exemplary embodiments.

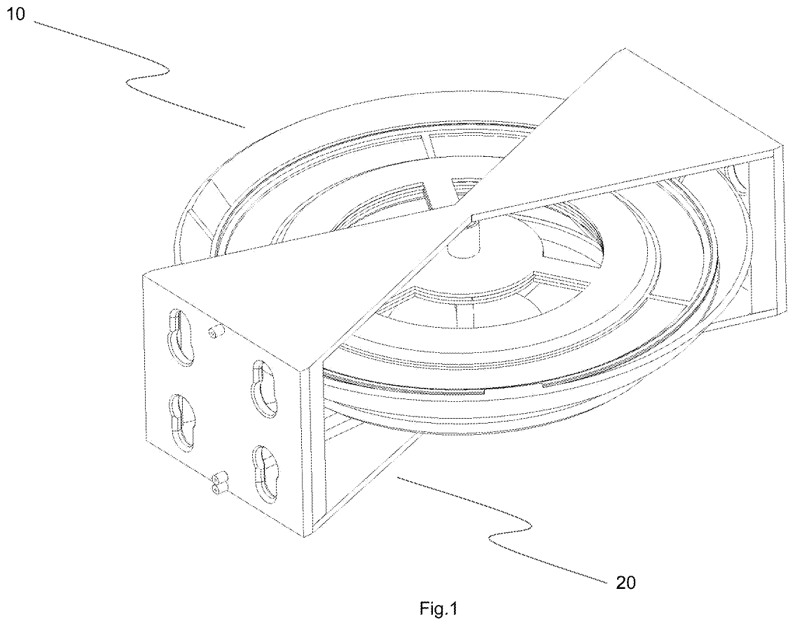

With reference to FIG. 1, provided is an overview of the toroidal bioreactor 10 (which may also be referred to as a bioreactor container, a bioreactor device, or a bioreactor assembly) housed in the frame 20.

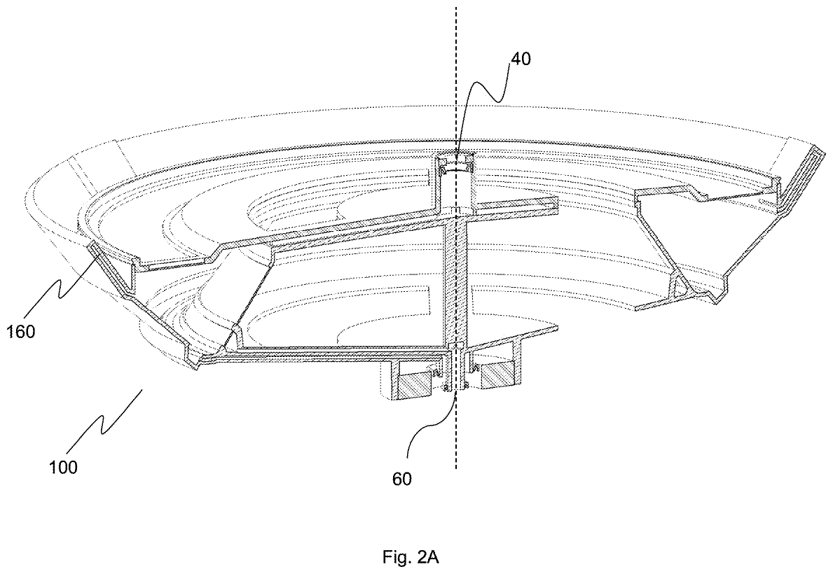

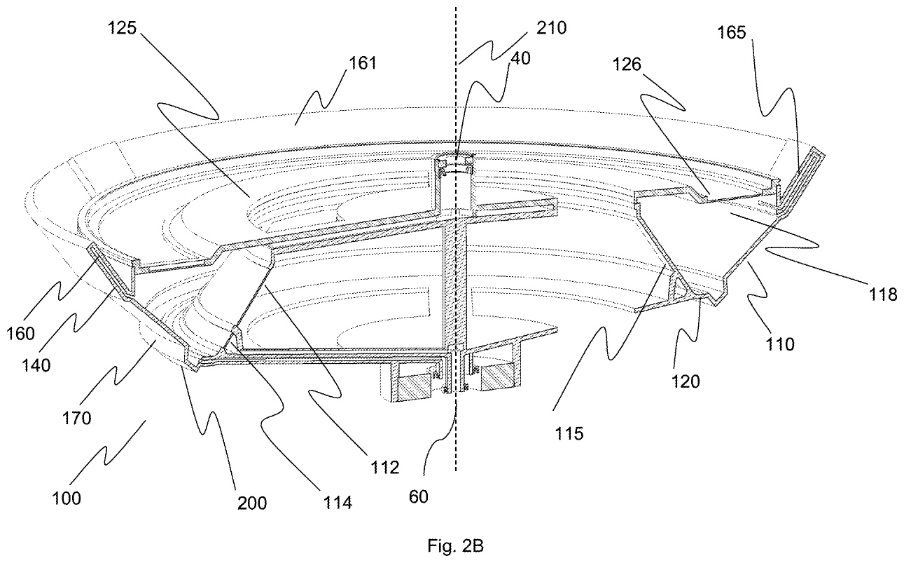

With reference to FIG. 2, the bioreactor container 100 may include plastic parts assembled together via methods and techniques known in the art, such as ultrasonic welding or bonding. Container 100 may further include membrane and filters suitable for the application (e.g., membranes and/or filters suitable for filtering beads, bacteria, cells, virus, or virus-like particles from a liquid media and/or for allowing gas exchange). The bioreactor container has slanted walls 110 and 115 connected by a bottom floor 120. Either of these surfaces can be composed of rigid plastics and/or fiber mesh 112 to allow the cells to exchange gasses and heat with the external air and to adhere to the membrane in case of adherent cells. The advantage of having slanted surfaces is that as the fluid amount increases, also the fluid-air surface increases. The angle of the slanted walls 110 and 115 relative to vertical can be chosen according to the need of the process and materials used, and could be varied also as function of the height of fluid. The walls 110 and 115 can thus be curved or have different sections vertically, for example to allow a more gentle increase of fluid-air surface area, with close to vertical surfaces, or a more drastic increase of air-exchange with shallow walls. This enables the bioreactor to allocate larger amounts of fluids, for example needed in allogeneic processes, starting from small amounts of fluid within the same container. The top area 118 is closed with a ceiling top surface 125. The top surface can be equipped with a filter 126 that allows exchange of gasses, such as oxygen, with the external atmosphere.

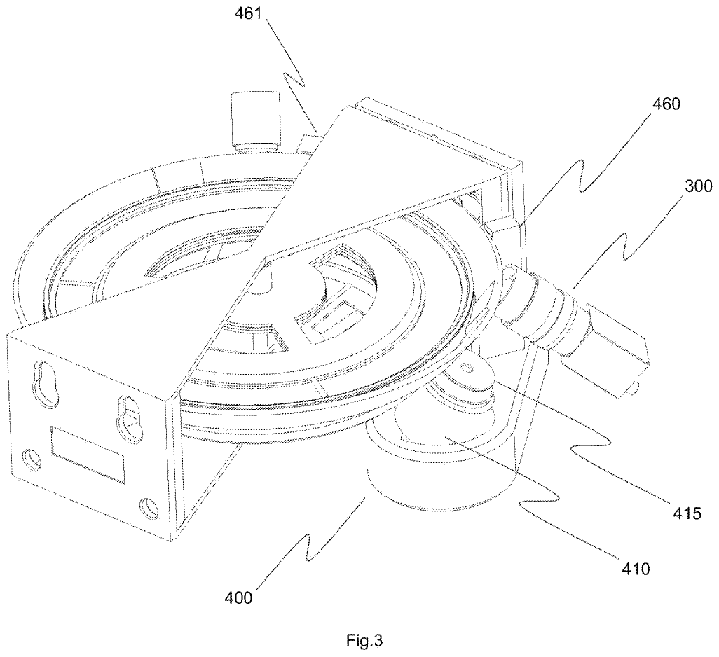

The container 100 is also equipped with a surface 200 which has provisions, such as teeth, protrusions or friction surfaces to be rotated along the axis 210 of the surface 200. This can be coincident with the axis of the toroidal container. The rotation is made possible by the nest 400 (also referred to herein as a drive mechanism), shown in FIG. 3, which has actuation means 410 with surfaces 415 matching to surface 200 that can rotate the container 100 along the axis 210. The rotation is monitored by optical sensors 460 and 461 that can track and measure the rotation of the contained 100 along the axis 210. These can be optical encoders tracking tacks or marks formed or imprinted on a corresponding area of the outset surface of the container 100. The use of two sensing areas allows redundancy as well as the measurement of the specific angle of rotation of the body 100 relative to the nest in order to identify the location of specific areas on the container body as it rotates along axis 210. Actuation means 410 can also be equipped with rotation sensors, such as incremental encoders, for controlling the rotation speed.

By controlling the speed of rotation, the cells can be exposed to a force in addition to gravity which pulls them more toward the external surface 110. Sinusoidal rotations are used to gently resuspend the cells. Different speeds of rotations can be used to distribute the mixture in the fluid according to their mass properties, for example when using beads or viral vectors. This allows the bioreactor to also perform other functions in addition to expansion, such as activation, transfection, enrichment and others.

The bioreactor is equipped with a surface 140 (e.g., part of a top edge of the container) which creates a cavity 160 (also referred to as micro cavity) in which the fluid with cells can be pushed by rotating the bioreactor over a certain minimum speed, called Minimum Sampling Speed (MSS). By controlling the time and speed pattern needed to reach the MSS, the user can control the fluid mixture and composition entering the cavity 160, selected based on the mass properties of the particles. With a quick acceleration after resuspension for example, viscosity effects would limit motion of the particles in the fluid based on their mass, while with a slower acceleration over time, the fluid entering the cavity 160 would mostly have the heavier, and/or lower viscosity, particles. The width and shape of the cavity 160 cross-section can be adjusted based on the properties of the fluid and particles of interest and can vary between different sections of the circumference, creating a selection by size and fluid properties for each section, in order to perform different functions.

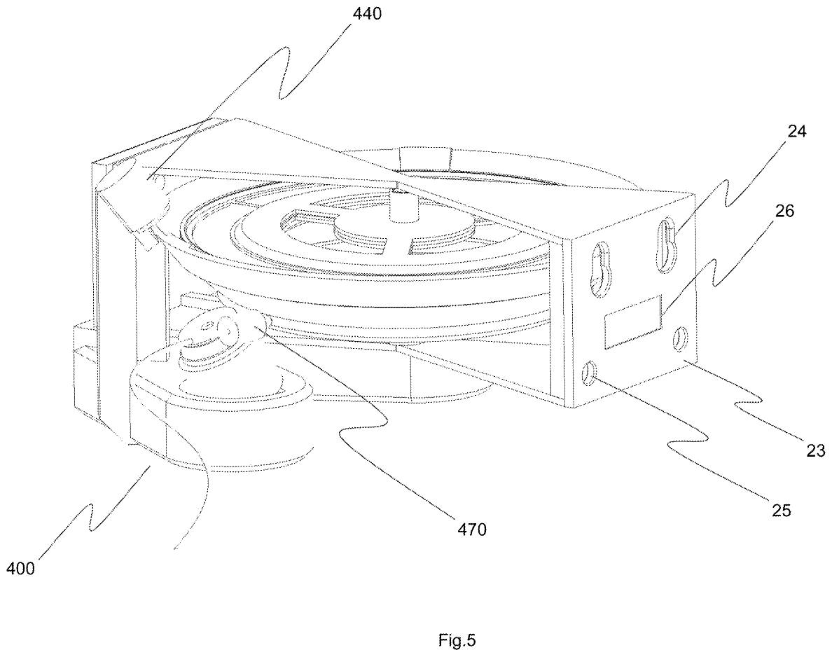

For example, with reference to FIG. 5, the nest 400 is equipped with a magnetic system 440 that can perform magnetic functions to specific sections of the cavities 160 as the bioreactor container 100 is rotated. These magnetic functions can be activation, beads handling and separation, magnetic transduction to mention a few. Sections of the surface 140 are made of a transparent or translucent material so that an image capturing system 300, shown in FIG. 3, positioned externally to the cavity 160 can capture high-resolution images of the fluid entering the cavity 160. The image capturing system 300 can be a high speed camera, microscope, or other imaging systems. The inner surface 161 opposite to the image capturing system can be coated with a reflective membrane or other filters to enable specific measurements and light capturing technologies in the system 300. In this way, several behaviors and key parameters of the particles in the fluid can be captured and observed without removing a sample and while keeping the system closed, thus preserving the cell culture from potential contamination risks and allowing in-line frequent measurements that can be digitally stored as time series of data.

The cavity 160 is connected to the central cavity 118 via channels 165 that ensure the same air pressure is maintained between cavity 160 and 118. Air filter 126 is connected to the cavity 118 and allows air exchange between the central cavity 118 and the incubator where the toroidal bioreactor is housed.

The container can be equipped with an optical surface 170 that allows sensors 470 housed in the nest (FIG. 5) to perform optical measurements on the cell growth such as standard optical sensors for pH, DO and other parameters that can be captured optically with an external detector placed radially, similarly to the imaging system 300.

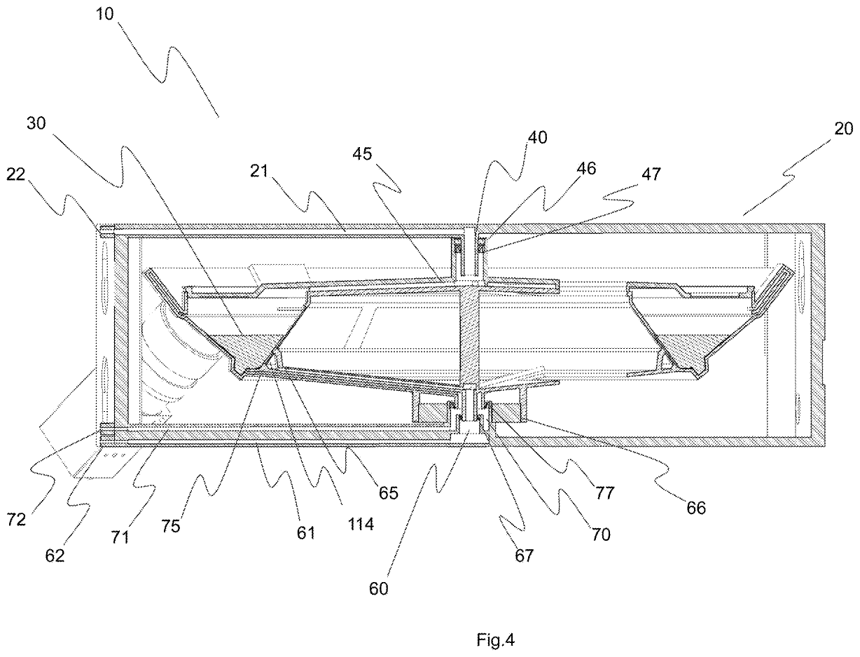

With reference to FIG. 4, depicted is a section of the bioreactor 10 including the frame 20, with fluid 30 inside the bioreactor container 100. The fluid is inserted in the container either manually, by removing the top plate 125 or in a closed manner via a central pivoting connector 40 which can rotate relative to the bioreactor container 100, mounted along the axis 210. The fluid inserted through the connector 40 reaches to the inside of the bioreactor container via one or multiple input channels 45.

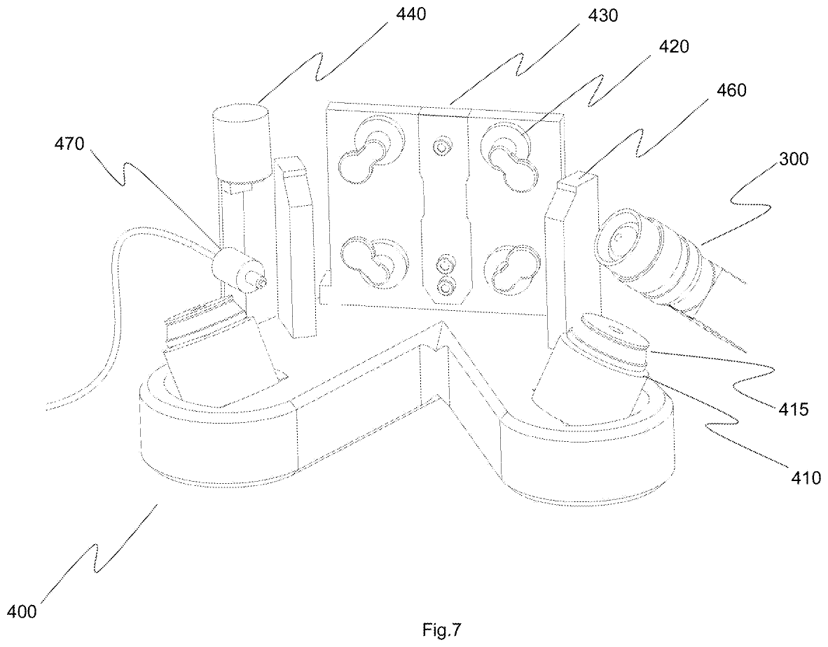

On the input side, the input channel 21 part of the frame 20 allows the fluid to reach connector 40 (i.e., a fluid inlet) from the external connector 22. Connector 22 is an aseptic-connection connector which mates with the corresponding receptacle in the nest 400 which will be described later in FIG. 7.

Through connector 22, fluid is delivered automatically such as the initial cell seeding, or other in-process fluids, such as media, reagents, and other fluids for either spot delivery or continuous perfusion.

Connector 40 is equipped with standard components such as rotating gaskets 47 and bearing surfaces 46 to allow it to perform its fluid connecting function between two rotating bodies without leaks, similar to a pivoting standard connector.

Fluid can also be removed though connectors 60 and 70 which are connected to the bottom of the container respectively with one more channels 65 and 75. These can be equipped with a filter 114 to maintain cells out, for example when used for the waste channel of perfusion, or used without a filter for harvesting or extra sampling. In this embodiment, connector 70 can be used for perfusion waste removal, and the filter 114 is placed on the inside of the bottom portion of the container 100. In this way, gentle rotations can be used to clear the filter from cells during perfusion and prevent clogging.

Similarly to connector 40, connectors 60 and 70 (i.e., fluid outlets) are pivoting fluid connectors, with standard means to allow them to perform their functions such as rotating gaskets 67 and 77, and bearing surfaces. Here a larger bearing system 66 supports both the pivoting connectors as well as takes radial and axial loads that are generated during the rotation of the container body 100 relative to the frame 20.

On the output side, the channels 61 and 71 part of the frame 20 allows the fluid coming respectively from connector 60 and 70 to reach the external connectors 62 and 72. These connectors, similar to connector 22, are aseptic-connection connectors which mate with the corresponding receptacles in the nest 400, part of a disposable set.

With reference again to FIG. 5, the handling plate 23 of the frame 20 is used for moving the bioreactor in and out of the nest. This is designed for automated robotics handling or manual handling. A tag 26 such as a barcode or QR code allows traceability by providing a unique ID as well as features for robotics image and pose recognition. Locking features 24 and centering features 25 allow a robotic gripper to precisely pick and place the bioreactor.

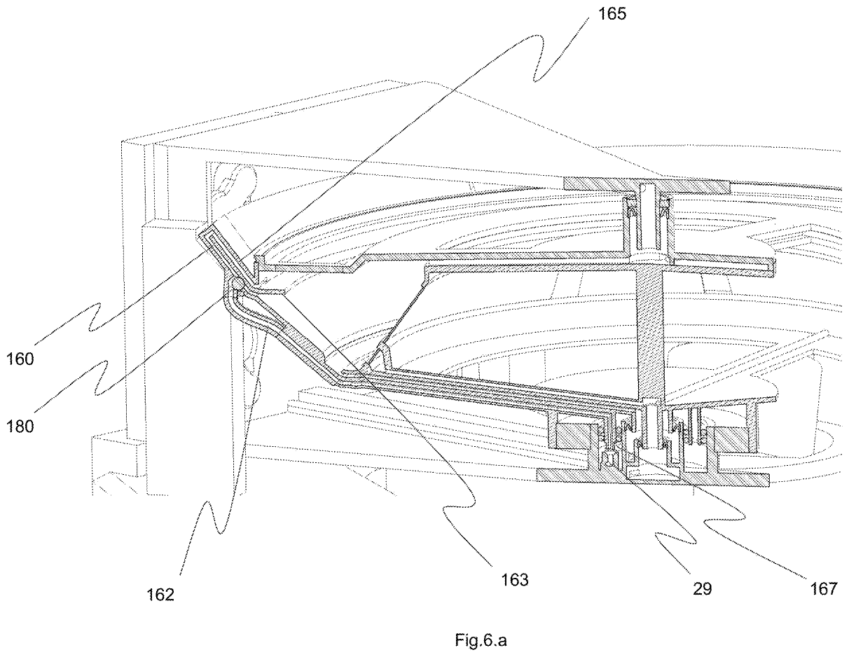

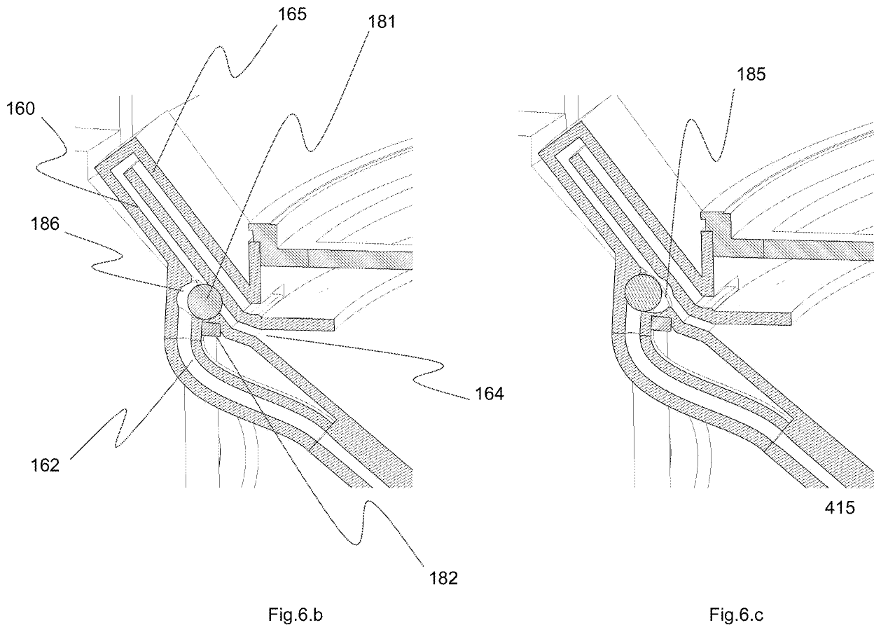

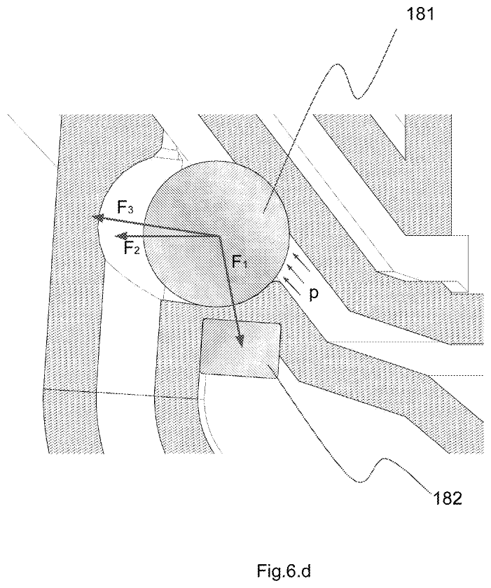

FIGS. 6A-6D depict an embodiment of a bioreactor container 100 with specialized sections of the side walls creating cavity 160 for the selection of small quantities of fluids, like the ones otherwise handled by pipetting. This enables the handling of small volumes of fluids (i.e. <1 ml), as well as larger volumes of fluids in the same closed system without the need to operate in an open-container fashion.

In this case the cavity 160 cross-section and length are sized appropriately to contain a set volume of fluid. Excess fluid would flow back in the container through the channel 165 described before. A buffle or screen 163 could facilitate channeling the fluid into the cavity 160 and prevent fluid from entering 165 inadvertently. A valve system 180 allows the selective opening of cavity 160 and the opening/closing of the output channel 162 which would deliver the selected amount of fluid outside of the container through a rotating connection. This can be similar to what described before or can be formed by the channels 167 and 29, respectively on the container body and the frame, that overlap at a specific angular rotation of the container body relative to the frame, as shown in FIG. 6.a. By controlling the air pressure in the container top area 118 via the input port 22 relative to the air pressure in the output channel 162, even small amounts of fluids can be moved through the channels, which would be coated with the state of the art coating and treatments to allow the fluid to easily advance the channel.

While this embodiment shows three output channels, either more outputs can be used in the same container, or a single central one can be used, thus simplifying the base of the container, for example in the case of a container used solely to dispense reagents in small volumes rather than for cell growth. In this case the toroidal bioreactor would be paired with reagents housed in the same shape containers, and connected through the consumables set.

When in need to deliver different amounts of fluids, multiple cavities 160 could be designed to have different set volumes and could be activated as needed by operating the respective valve systems 180.

FIGS. 6B-6D show an embodiment of the valve system 180 where a moving body 181, in this case a spherical body, selectively closes the fluid input channel 164 or the output channel 162 by sitting respectively in the sealing area 185 or 186, thereby functioning as a valve mechanism. The moving body 181 has a preferred resting position in the sealing area 185. This equilibrium position is held by a force that can be created by a spring, an elastic element or by connecting the moving body 181 to the body of the container with a live hinge or preloaded plastic piece. In this embodiment, the spherical body 181 is composed of a ferromagnetic core material that is attracted by a magnet 182 in its resting position against surface 185, closing the channel 164. When there is the need to extract and dispense the small volume of fluid, the container is spinned as described before. The rotation of the container body causes the fluid to push against the moving body 181 under centrifugal force (an actuation force), creating a pressure p that acts against the sealing force of channel 164. The body 181 itself is also subject to the centrifugal force, until channel 164 is opened and the fluid can fill the cavity 160. Conversely, the moving body 181 moving radially outward also closes the output channel 162. This is a second equilibrium position of the moving body 181 against the output sealing surface 186, subject to the centrifugal force and pressure p. Gravity is omitted for simplicity. Once the centrifugal force is reduced or stopped, the moving body 181 would return to the first resting position closing again the input channel 164 and opening the output channel 162 thus dispensing the fluid that occupied the cavity 160 to the output channel. In some embodiments, the (at least one) micro cavity feature is configured to sample an aliquot of the fluid contained within the toroidal container by increasing an angular velocity for rotation of the toroidal container about its axis so that the angular velocity exceeds a specified minimum angular velocity threshold.

FIG. 6D shows an example of the forces on the moving body 181 in this embodiment. In this case F1 is the force keeping the ball 181 against the surface 185 thanks to the attraction to magnet 182. The fluid pressure is indicated as p, while the centrifugal force is indicated as F2. In this embodiment an additional actuation force F3 is used to make the ball snap quickly between the two equilibrium positions, thus creating a true bi-stable mechanism that works in conjunction with the spinning of the container body. The actuation force F3 is a magnetic force generated by an electromagnet situated in the nest, similar to 440 shown before. In order to quickly open channel 164, the force F3 snaps the ball out of the first resting position, i.e. against surface 185, into the second resting position, i.e. against surface 186. Here the pressure and the centrifugal force overcome the force F2, which is inversely proportional to the distance between the ball 181 and the magnet 182. In this position force F3 is not needed anymore but can be kept for additional design margin. Since the container body is rotating, and the electromagnet is localized in the nest, the force F3 will be seen as pulsating by the ball 181. As soon as the rotational speed is reduced, and F3 turned off, the ball is magnetically attracted back by magnet 182 in the initial position. This can happen by design at a speed higher than MSS, i.e. a rotational speed that still maintains the fluid in the cavity 160.

While in this embodiment the moving body 181 acts as a valve to open and close both the input channel 164 and output channel 162, it is possible to have a moving body for each channel, for example with a flap of materials or spring-loaded body for each channel that are subject to similar forces thus obtaining the same results.

FIG. 7 shows the main components of nest 400, which have been previously described. The bioreactor container is secured to the nest through features 24 in the frame 20 that mate with locking features 420 to secure the bioreactor in place. Actuation means 410, such as servo motors, rotate the bioreactor container 100 via couplings 415, which can be friction surfaces, gears or systems to transmit motion. Encoders 460 and 461 measure the rotation of the bioreactor container. The imaging system 300 is used to observe the fluid and its particles, and optical sensor 470 captures many of the key parameters such as pH, DO and others. The system 440 is a magnetic actuator that can be used for specialized purposes, such as transduction of the cells in the cavities 160, magnetic beads processing and removal, and other applications. A system similar to 440, such as an electromagnet, can also be included for operating the valve system 180 to dispense small amounts of fluids. The disposable cartridge 430 houses the mating aseptic connectors that engage with the corresponding ports on the bioreactor frame. These can be needle-free ports with rotating luer lock, push-pull or lock-tab connectors, needle and membrane connectors or other fluid connectors used for these applications. Multiple of these nest can be located in a bioreactor that controls the environmental parameters, such as temperature, humidity, CO2, pressure. The disposable cartridge 430 is connected to a set of reagents through tubings operated by pinch valves in order to dispense the desired amounts of fluids per the prescribed protocol, such as perfusion feed for example. These reagents can also be contained in simplified toroidal containers 100 for the delivery of small amounts of fluids, for example in the case of cytokines or CRISPR reagents, as described before.

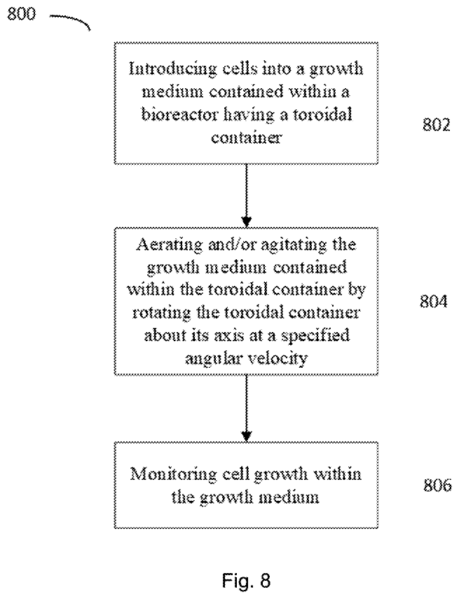

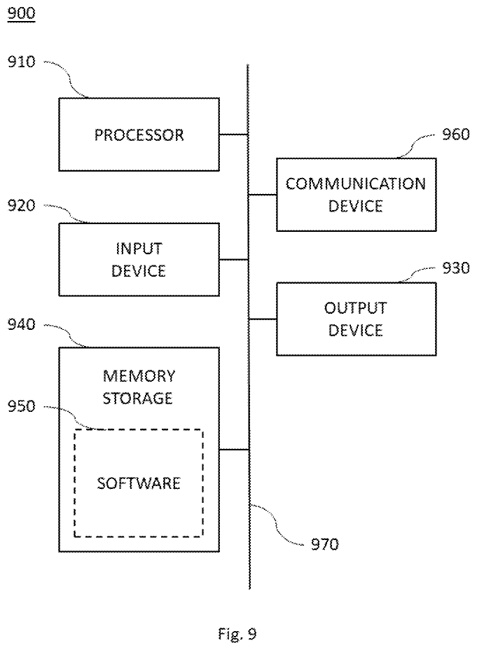

In other aspects, with reference to FIG. 8, provided is an exemplary method 800 of using the bioreactors described herein. In step 802, cells are introduced in a growth medium contained with any of the bioreactors described herein, which have a toroidal container. In step 804, the growth medium is aerated and/or agitated contained within the toroidal container by rotating the toroidal container about its axis at a specified angular velocity. In step 806, cell growth within the grown medium is monitored by any suitable methods, including the methods of monitoring described herein. For example, in one variation, the cell growth is monitored by performing high speed imaging of cells suspended in the growth medium or pushed by the centrifugal force inside the cavities 160. In another variation, cell growth is monitored by performing high speed imaging of cells attached to an interior surface of the first side wall of the toroidal container. In another variation, cell growth within the growth medium is monitored by performing high speed imaging of the cells and fluids contained in the micro-cavities designed for imaging, such as micro-cavities fabricated from optically transparent materials such as PC. In yet another variation, cell growth is monitored by sampling an aliquot of the growth medium using the at least one micro cavity feature by increasing an angular velocity for rotation of the toroidal container so that it exceeds a specified minimum angular velocity threshold, and delivering the aliquot of the growth medium to a stationary external sample container for testing. In yet another variation, rotation for cell resuspension and clustering prevention are employed.

In one aspect, provided is method for culturing cells, the method comprising:
introducing cells into a growth medium contained within a bioreactor comprising:
   a toroidal container having a cross-sectional profile comprising a top plate, a first side-wall, and a second side-wall, and
   at least one micro cavity feature integrated with a top edge of the first side-wall and configured to:
   (i) sample an aliquot of fluid contained within the toroidal container when a force is applied to a valve mechanism integrated with the at least one micro cavity feature; and
   (ii) deliver the fluid aliquot to a stationary external fluid sample container;
aerating and/or agitating the growth medium contained within the toroidal container by rotating the toroidal container about its axis at a specified angular velocity; and
monitoring cell growth within the growth medium by at least one of:
   performing high speed imaging of cells suspended in the growth medium,
   performing high speed imaging of cells attached to an interior surface of the first side wall of the toroidal container, and/or
   performing high speed imaging of the cells and fluids contained in micro-cavities designed for imaging, such as micro-cavities fabricated from an optically transparent materials, such as PC.
   sampling an aliquot of the growth medium using the at least one micro cavity feature by increasing an angular velocity for rotation of the toroidal container so that it exceeds a specified minimum angular velocity threshold, and delivering the aliquot of the growth medium to a stationary external sample container for testing.

In some embodiments, the method further comprises monitoring at least one of pH, dissolved oxygen (DO), and temperature within the growth medium as a function of time.

In some embodiments, prior to the sampling step, an angular velocity for rotation of the toroidal container is adjusted to select a subset of cells for sampling by the at least one micro cavity feature.

In some embodiments, prior to the sampling step, an external force is applied to select a subset of cells for sampling by the at least one micro cavity feature.

In certain embodiments, the cells are attached to magnetic beads and the external force is a magnetic force.

Definitions: Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly indicates otherwise. Any reference to “or” herein is intended to encompass “and/or” unless otherwise stated, and encompasses any and all possible combinations of one or more of the associated listed items.

As used herein, the terms “includes, “including,” “comprises,” and/or “comprising” specify the presence of stated features, integers, steps, operations, elements, components, and/or units but do not preclude the presence or addition of one or more other features, integers, steps, operations, elements, components, units, and/or groups thereof.

Throughout this application, various parameter values may be presented in a range format. It should be understood that the description in range format is merely for convenience and brevity, and should not be construed as an inflexible limitation on the scope of the disclosure. Accordingly, the description of a range should be considered to have specifically disclosed all possible subranges as well as individual numerical values within that range, irrespective of whether a specific numerical value or specific sub-range is expressly stated. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 1.4, 2, 3, 3.6, 4, 5, 5.8, and 6. This applies regardless of the breadth of the range.

Polymeric materials: The disclosed bioreactor containers may be fabricated from any of a variety of materials (or combinations thereof) known to those of skill in the art including, but not limited to, glass (e.g., borosilicate glass, soda lime glass, etc.), fused silica (quartz), polymer (e.g., polystyrene (PS), tissue culture-treated polystyrene (TCPS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET), polydimethylsiloxane (PDMS), etc.), polyetherimide (PEI) and perfluoroelastomer (FFKM) as more chemically inert alternatives, or any combination thereof. FFKM is also known as Kalrez.

Fabrication techniques: The disclosed bioreactor containers may be fabricated using any of a variety of techniques (or combinations thereof) known to those of skill in the art, where the choice of fabrication technique is often dependent on the choice of material used, and vice versa. Examples of suitable fabrication techniques include, but are not limited to, injection molding, casting, extrusion, drawing, precision computer numerical control (CNC) machining and boring, laser photoablation, micro-molding, embossing, 3D-printing, thermal bonding, ultrasonic welding, adhesive bonding, anodic bonding, and the like.

Optical sensing and/or imaging modules: The disclosed bioreactor containers and/or systems may comprise optical sensing and/or imaging instrumentation. In some embodiments, the optical sensing and/or imaging instrumentation may comprise one or more light sources, one or more objective lenses, one or more image sensors or cameras, one or more processors or controllers, one or more additional optical components (e.g., lenses, mirrors, prisms, beam-splitters, optical filters, colored glass filters, narrowband interference filters, broadband interference filters, dichroic reflectors, diffraction gratings, apertures, shutters, optical fibers, optical waveguides, acousto-optic modulators, and the like), or any combination thereof. In some instances, the optical sensing and/or imaging instrumentation may comprise a focus mechanism, e.g., an autofocus mechanism. In some instances, the optical sensing and/or imaging instrumentation may be configured to perform multichannel imaging, e.g., multichannel fluorescence imaging comprising the use of excitation light at one or more excitation wavelengths, and imaging the emitted fluorescence at two or more different emission wavelengths. In some instances, the optical sensing may comprise a laser emitter and fiber optics sensors, fiber optics grating or other laser-based measuring systems.

Fluidics modules and components: The disclosed bioreactor containers and/or systems may comprise one or more fluidics modules (or fluidics controllers) configured to control the delivery of fluids (e.g., gasses and/or liquid) to the internal volume of the toroidal container. In some embodiments, the one or more fluidics controllers may be configured to control volumetric flow rates for one or more gasses and/or liquids, linear flow velocities for one or more gasses and/or liquids, mixing ratios for one or more gasses and/or liquids, or any combination thereof. Fluid flow may be generated by pressure differences in the respective containers, by adding or removing gas (air), or by fluid pumps. Fluidics modules may comprise one or more gas and/or liquid flow sensors (e.g., flow rate sensors, pressure sensors, etc.), pressure sensors, one or more gas and/or liquid flow actuators (e.g., pumps), one or more fluid flow control devices (e.g., valves), one or more processors (and associated electronics), tubing and connectors to connect the one or more fluidics modules to one or more toroidal containers, or any combination thereof.

Temperature control modules: The disclosed bioreactor containers and systems may comprise one or more temperature control modules (or temperature controllers) configured to maintain a specified temperature for the gas and liquid within one or more toroidal containers for the purpose of facilitating the accuracy and reproducibility of cell growth and/or analysis results. Examples of temperature control components that may be incorporated into the disclosed bioreactor containers and/or systems and controlled by a temperature control module include, but are not limited to, resistive heating elements, infrared light sources, Peltier heating or cooling devices, heat sinks, thermistors, thermocouples, and the like.

In some instances, the temperature control module may provide for a programmable temperature change at a specified, adjustable time prior to performing specific assay or analysis steps. In some instances, the temperature control module may provide for programmable changes in temperature over specified time intervals. In some instances, the temperature control module may further provide for cycling of temperatures between two or more set temperatures with specified frequency and ramp rates.

Motion control modules: The disclosed bioreactor containers and/or systems may comprise one or more motion control modules (or motion controllers) configured to control the angular velocity of one or more toroidal containers and/or the position of one or more toroidal containers relative to an optical sensor, relative to an objective lens of an imaging module objective lens, etc. In some instances, the motion control module may control the position of the toroidal container in one dimension, two dimensions, or three dimensions (e.g., in the X-, Y-, and/or Z-directions). In some instances, the motion control module may separately or additionally control a degree of rotation of the toroidal container in one, two, or three dimensions. In some instances, the motion control module may be interfaced with an imaging module to also provide control of an autofocus mechanism. For example, the motion control module may be configured to adjust the focal plane by moving the toroidal container and/or by moving an objective lens (or other optical component) of the imaging module.

In some instances, the motion control module may comprise one or more (e.g., one, two, three, or more than three) translation stages, one or more (e.g., one, two, three, or more than three) rotational stages, one or more (e.g., one, two, three, or more than three) linear encoders, one or more (e.g., one, two, three, or more than three) rotary encoders, associated motors and control electronics, or any combination thereof. In some instances, the motion control module may further control components of the imaging module such as an automated objective lens turret or slide, or a microscope turret-mounted focus adjustment mechanism.

Suitable translation stages are commercially available from a variety of vendors, for example, Parker Hannifin, Harmonic Drives, Kollmorgen, Wittenstein, Ametek, THK, Applied motion and others. Precision translation stage systems typically comprise a combination of several components including, but not limited to, linear actuators, optical encoders, servo and/or stepper motors, and motor controllers or drive units. High precision and repeatability of stage movement is required for the disclosed bioreactor containers and/or systems in order to ensure accurate and reproducible fluid control, positioning, and optical sensing and/or imaging.

System control module: The disclosed bioreactor containers and/or systems may comprise one or more system control modules (or system controllers) configured to synchronize and control data communication between other functional units of the system, e.g., the one or more optical sensing and/or imaging modules, one or more fluidics modules, one or more temperature control modules, one or more motion control modules, or any combination thereof. In some instances, a system control module may comprise one or more processors, one or more power supplies, one or more wired and/or wireless data communication interfaces, one or more memory storage devices, one or more user interface devices, or any combination thereof. In some instances, the system control function may be provided by an external computer or computer system. In some instances, the one or more system control modules may interface with one or more external computers or computer systems.

Computing devices and systems: FIG. 9 illustrates an example of a computing device or system in accordance with one or more examples of the disclosure. Device or system 900 can be a host computer connected to a network. Device or system 900 can be a client computer or a server. As shown in FIG. 9, device or system 900 can be any suitable type of microprocessor-based device, such as a personal computer, workstation, server, or handheld computing device (portable electronic device), such as a phone or tablet. The device can include, for example, one or more of processor 910, input device 920, output device 930, storage 940, and communication device 960. Input device 920 and output device 930 can generally correspond to those described above, and they can either be connectable or integrated with the computer.

Input device 920 can be any suitable device that provides input, such as a touch screen, keyboard or keypad, mouse, or voice-recognition device. Output device 930 can be any suitable device that provides output, such as a touch screen, haptics device, or speaker.

Storage 940 can be any suitable device that provides storage, such as an electrical, magnetic, or optical memory including a RAM, cache, hard drive, or removable storage disk. Communication device 960 can include any suitable device capable of transmitting and receiving signals over a network, such as a network interface chip or device. The components of the computer can be connected in any suitable manner, such as via a physical bus 970 or wirelessly.

Software 950, which can be stored in memory/storage 940 and executed by processor 910, can include, for example, the programming that embodies the functionality of the present disclosure (e.g., as embodied in the devices described above).

Software 950 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 940, that can contain or store programming for use by or in connection with an instruction execution system, apparatus, or device.

Software 950 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device or system 900 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device or system 900 can implement any operating system suitable for operating on the network. Software 950 can be written in any suitable programming language, such as C, C++, Java, or Python. In various embodiments, application software embodying the functionality of the present disclosure can be deployed in different configurations, such as in a client/server arrangement or through a web browser as a web-based application or web service, for example.

### ENUMERATED EMBODIMENTS

The following enumerated embodiments are representative of some aspects of the invention.

A1. A toroidal bioreactor system 10 for growing, expanding, activating, transducing and manipulating cells in a closed system, comprising:
container 100 configured to (i) rotate around a vertical axis 210, and (ii) receive fluid 30, wherein the container comprises;
   a first interior slanted wall 110 and a second interior slanted wall 115 forming a v-shape central cavity 118 positioned around the vertical axis, wherein the exterior and interior slanted walls are connected by a bottom floor 120 and the top area of cavity 118 closed with a ceiling top surface 125,
   a nest 400 configured to rotate the container 100 along the axis 210, having an actuator 410 with surfaces 415 matching to surface 200 that causes rotation of the container.

A2. The system of embodiment A1, wherein each of the first interior slanted wall and the second interior slanted wall is independently composed of rigid plastics and/or fiber mesh 112 to allow the cells to exchange gasses and heat with external air and to adhere to the membrane in case of adherent cells.

A3. The system of embodiment A1 or A2, wherein the first interior slanted wall 110 and the second interior slanted wall slanted walls 115 have an angle relative to vertical that is varied as function of the height of fluid.

A4. The system of any one of embodiments A1 to A3, wherein each of the first interior slanted wall 110 and the second interior slanted wall slanted walls 115 is independently curved.

A5. The system of any one of embodiments A1 to A4, wherein the first interior slanted wall 110 and the second interior slanted wall slanted walls 115 independently have different sections vertically to allow a more gentle increase of fluid-air surface area, with close to vertical surfaces, or a more drastic increase of air-exchange with shallow walls.

A6. The system of any one of embodiments A1 to A5, wherein the top surface is equipped with a filter 126 confirmed for exchange of gasses, such as oxygen, with external atmosphere.

A7. The system of any one of embodiments A1 to A5, wherein container 100 further comprises a surface 200 which has provisions (e.g., teeth, protrusions or friction surfaces) to be rotated along the axis 210 of the surface 200.

A8. The system of embodiment 6, wherein the surface with provisions is coincident with the axis of the container.

A9. The system of any one of embodiments A1 to A8, further comprising optical sensors 460 and 461 configured to track and measure the rotation of the container 100 along the axis 210.

A10. The system of embodiment A9, wherein the optical sensors are optical encoders configured to track tacks or marks formed or imprinted on a corresponding area of the outset surface of the container 100.

A11. The system of embodiment A9 or A10, wherein the optical sensors are configured to measure the specific angle of rotation of the container 100 relative to the nest in order to identify the location of specific areas on the container 100 as it rotates along axis 210.

A12. The system of any one of embodiments A1 to A11, wherein actuator 410 is further equipped with rotation sensors, such as incremental encoders, for controlling the rotation speed.

A13. The system of embodiment A12, wherein the rotation sensors are configured to (i) adjust the rotation speed so as to expose cells to a force in addition to gravity which pulls them more toward the external surface 110 and/or to distribute the mixture in the fluid according to their mass properties; (ii) rotate in a sinusoidal form to gently resuspend the cells.

A14. The system of any one of embodiments A1 to A13, wherein the container further comprises a surface 140 connected to exterior slanted wall, which creates a cavity 160 in which the fluid with cells can be pushed by rotating the bioreactor over a Minimum Sampling Speed (MSS).

A15. The system of embodiment A14, wherein the cavity 160 is connected to the central cavity 118 via channels 165 that ensure the same air pressure is maintained between cavities 160 and 118.

A16. The system of embodiment A14 or A15, wherein nest 400 is equipped with a magnetic system 440 confirmed to perform magnetic functions to specific sections of the cavities 160 as the container 100 is rotated.

A17. The system of any one of embodiments A14 to A16, wherein sections of the surface 140 are made of a transparent or translucent material.

A18. The system of any one of embodiments A14 to A17, wherein the bioreactor system 10 further comprises an image capturing system 300 positioned externally to the cavity 160, configured to capture high-resolution images of the fluid entering the cavity 160.

A19. The system of embodiment A18, wherein the image capturing system 300 is a high speed camera, microscope, or other imaging systems.

A20. The system of embodiment A18 or A19, wherein cavity 160 has an inner surface 161 opposite to the image capturing system.

A21. The system of embodiment A20 wherein inner surface 161 is coated with a reflective membrane or other filters to enable specific measurements and light capturing technologies in the system 300.

A22. The system of any one of embodiments A1 to A21, wherein the container further comprises air filter 126 is connected to the cavity 118, configured for air exchange between the central cavity 118 and the incubator where the bioreactor system 10 is housed.

A23. The system of any one of embodiments A1 to A22, wherein the container is further equipped with an optical surface 170 that allows sensors 470 housed in the nest to perform optical measurements on the cell growth such as standard optical sensors for pH, DO and other parameters that can be captured optically with an external detector placed radially, similarly to the imaging system 300.

A24. The system of any one of embodiments A1 to A23, wherein the bioreactor system 10 further comprises a frame 20.

A25. The system of any one of embodiments A1 to A24, wherein the fluid is inserted into the container manually.

A26. The system of embodiment A25, wherein the fluid is inserted into the container by removing the ceiling top surface 125.

A27. The system of any one of embodiments A1 to A24, wherein the fluid is inserted into the container in a closed manner via a central pivoting connector 40, configured to rotate relative to the container 100, mounted along the axis 210, wherein the fluid inserted through the connector 40 reaches to the inside of the container via one or multiple input channels 45.

A28. The system of any one of embodiments A1 to A27, wherein frame 20 further comprises input channel 21, configured to allow the fluid to reach connector 40 from an external connector 22.

A29. The system of embodiment 28, wherein connector 22 is an aseptic-connection connector that mates with the corresponding receptacle in the nest 400.

A30. The system of any one of embodiments A27 to A29, wherein connector 40 is configured to perform its fluid connecting function between two rotating bodies without leaks.

A31. The system of any one of embodiments A27 to A30, wherein connector 40 is equipped with rotating gaskets 47 and/or bearing surfaces 46.

A32. The system of any one of embodiments A1 to A31, wherein frame 20 further comprises connectors 60 and 70 that are connected to the bottom of the container respectively with one more channels 65 and 75.

A33. The system of embodiment A32, wherein connectors 60 and 70 are equipped with a filter 114 to maintain cells out.

A34. The system of embodiment A32, wherein connectors 60 and 70 are equipped with rotating gaskets 67 and 77, and/or bearing surfaces.

A35. The system of any one of embodiments A1 to A34, wherein frame 20 further comprises channels 61 and 71, configured to allow allows the fluid coming respectively from connector 60 and 70 to reach the external connectors 62 and 72.

A36. The system of embodiment A35, wherein connectors 61 and 71 are aseptic-connection connectors configured to mate with the corresponding receptacles in the nest 400.

A37. The system of any one of embodiments A1 to A36, wherein frame 20 further comprises a handling plate 23, configured for moving the bioreactor in and out of the nest.

A38. The system of embodiment A37, wherein handling plate 23 comprises tag 26 such as a barcode or QR code allows traceability by providing a unique ID as well as features for robotics image and pose recognition.

A39. The system of embodiment A37 or A38, wherein handling plate 23 comprises locking features 24 and centering features 25, configured to allow a robotic gripper to pick and place the bioreactor.

A40. The system of any one of embodiments A14 to A39, wherein cavity 160 is configured to receive and contain a volume of fluid by pipetting in a closed-container fashion.

A41. The system of embodiment A40, wherein cavity 160 is connected to channel 165, configured so that excess fluid flows back into the container via channel 165.

A42. The system of any one of embodiments A1 to A41, wherein the container further comprises screen 163, configured to facilitate channeling the fluid into the cavity 160 and prevent fluid from inadvertently entering channel 165.

A43. The system of any one of embodiments A1 to A42, wherein the container further comprises a valve system 180, configured for the selective opening of cavities 160 and the opening/closing of the output channel 162 which would deliver the selected amount of fluid outside of the container through a rotating connection.

A44. The system of embodiment A43, wherein the valve system 180 is further connected to the channels 167 and 29, respectively on the container 100 and the frame 20, that overlap at a specific angular rotation of the container body relative to the frame.

A45. The system of any one of embodiments A1 to A44, wherein the air pressure in the container top area of cavity 118 is controlled via the input port 22 relative to the air pressure in the output channel 162, configured to move volumes of fluids through the channels.

A46. The system of any one of embodiments A1 to A45, wherein the container comprises one or more additional cavities 160, configured to receive and contain different amounts of fluids, wherein the one or more additional cavities 160 are independently activated by operating the respective valve systems 180.

A47. The system of any one of embodiments A1 to A46, wherein the valve system 180 further comprises a moving body 181, configured to selectively close the fluid input channel 164 or the output channel 162 by sitting respectively in the sealing area 185 or 186.

A48. The system of embodiment A47, wherein the moving body 181 has an equilibrium position in the sealing area 185, and this equilibrium position is held by a force that can be created by a spring, an elastic element or by connecting the moving body 181 to the body of the container with a live hinge or preloaded plastic piece.

A49. The system of any one of embodiments A1 to A48, wherein the nest 400 further comprises locking features 420 that secure container 100 to the nest through locking features 24 in the frame 20 that mate with locking features 420.

A50. The system of any one of embodiments A1 to A49, wherein the actuator 440 is a magnetic actuator, configured for transduction of the cells in the cavities 160, magnetic beads processing and removal, and other applications.

The following enumerated embodiments are representative of other aspects of the invention.

B1. A bioreactor comprising:
a toroidal container having a cross-sectional profile comprising a top plate, a first side-wall, and a second side-wall;
at least one fluid inlet configured to provide fluid communication between a stationary external fluid source and an interior of the toroidal container as the toroidal bioreactor rotates about its axis; and
at least one fluid outlet configured to provide fluid communication between the interior of the toroidal container and a stationary exterior fluid reservoir as the toroidal bioreactor rotates about its axis.

B2. The bioreactor of embodiment B1, further comprising at least one micro cavity feature integrated with the first side-wall and configured to sample an aliquot of fluid contained within the toroidal container.

B3. The bioreactor of embodiment B2, wherein the at least one micro cavity feature is integrated with a top edge of the first side-wall.

B4. The bioreactor of embodiment B2 or embodiment B3, wherein the at least one microcavity feature is configured to sample an aliquot of fluid contained within the toroidal container when an actuation force is applied to the at least one micro cavity feature.

B5. The bioreactor of any one of embodiments B2 to B4, wherein the at least one micro cavity feature is configured to deliver the fluid aliquot to a stationary external fluid sample container.

B6. The bioreactor of any one of embodiments B2 to B5, wherein the at least one micro cavity feature comprises:
a sample fluid inlet configured to provide fluid communication between an interior volume of the micro cavity feature and a fluid contained within the toroidal container;
a valve mechanism integrated with the sample fluid inlet and configured to open when an actuation force is applied;
the interior volume of the micro cavity feature;
a return fluid outlet configured to vent air and/or excess fluid from the interior volume of the micro cavity feature; and
a sample fluid outlet configured to provide fluid communication between the interior volume of the micro cavity feature and a stationary external fluid sample container.

B7. The bioreactor of embodiment B6, wherein the interior volume of the at least one micro cavity feature is at least 5 microliters, 10 microliters, 20 microliters, 30 microliters, 40 microliters, 50 microliters, 60 microliters, 70 microliters, 80 microliters, 90 microliters, 0.1 milliliters, 0.2 milliliters, 0.3 milliliters, 0.4 milliliters, 0.5 milliliters, 0.6 milliliters, 0.7 milliliters, 0.8 milliliters, 0.9 milliliters, 1 milliliters, 2 milliliters, 3 milliliters, 4 milliliters, or 5 milliliters.

B8. The bioreactor of embodiment B6 or embodiment B7, wherein the valve mechanism of the at least one micro cavity feature comprises a moveable component configured to toggle the valve between:
(i) an open sample fluid inlet/closed sample fluid outlet configuration, and
(ii) a closed sample fluid inlet/open sample fluid outlet configuration, upon application of the actuation force.

B9. The bioreactor of any one of embodiments B4 to B8, wherein the actuation force comprises a centrifugal force applied when an angular velocity for rotation of the toroidal container about its axis exceeds a specified minimum angular velocity threshold.

B10. The bioreactor of any one of embodiments B4 to B9, wherein the actuation force comprises a magnetic force.

B11. The bioreactor of any one of embodiments B8 to B10, wherein the moveable component comprises a rolling element that rolls between a first indent and a second indent, wherein the first indent and second indent correspond to the open sample fluid inlet/closed sample fluid outlet configuration and the closed sample fluid inlet/open sample fluid outlet configuration, respectively.

B12. The bioreactor of any one of embodiments B1 to B11, wherein:
the intersection of the first side-wall with a first imaginary plane parallel to a plane of the top plate defines a first diameter of the toroidal container;
wherein the intersection of the second side-wall with a second imaginary plane parallel to the plane of the top plate defines a second diameter of the toroidal container; and wherein the second diameter is smaller than the first diameter.

B13. The bioreactor of embodiment B12, wherein the first diameter ranges from about 2 cm to about 35 cm.

B14. The bioreactor of embodiment B12 or embodiment B13, wherein the second diameter ranges from about 1 cm to about 30 cm.

B15. The bioreactor of any one of embodiments B1 to B14, wherein the first side-wall and the second side-wall are slanted so as to converge at a third imaginary surface that defines an angle between the first side-wall and the second side-wall

B16. The bioreactor of any one of embodiment B1 to B15, wherein at least one of the first side-wall or the second-side wall is curved rather than planar.

B17. The bioreactor of any one of embodiments B1 to B16, further comprising a bottom plate that intersects with the first side-wall and the second side-wall and is parallel to the top plate.

B18. The bioreactor of any one of embodiments B1 to B17, wherein at least a portion of the top plate, the first side-wall, and/or the second side-wall is composed of a gas permeable membrane.

B19. The bioreactor of any one of embodiments B1 to B18, wherein the first side-wall is an exterior side-wall, and wherein at least a portion of the first side-wall is optically transparent and configured to enable high speed imaging of particles suspended in a fluid contained within the toroidal container as the toroidal container rotates about its axis.

B20. The bioreactor of any one of embodiments B1 to B19, wherein the first side-wall is an exterior side-wall, and wherein at least a portion of the first side-wall is optically transparent and configured to enable high speed imaging of particles attached to an interior surface of the first side-wall as the toroidal container rotates about its axis.

B21. The bioreactor of any one of embodiments B1 to B20, wherein the first side-wall is an exterior side-wall, and wherein at least a portion of the first side-wall is optically transparent and configured to enable optical sensing of at least one of pH, dissolved oxygen (DO), O2, N2, CO2, metabolomic profile, glutamate, glutamine, lactate, glucose, volume, flow rate, pressure, and temperature in a fluid contained within the toroidal container as the toroidal container rotates about its axis.

B22. The bioreactor of any one of embodiments B19 to B21, wherein the particles comprise cells, virus particles, beads, or any combination thereof

B23. The bioreactor of any one of embodiments B15 to B22, wherein the angle between the first side-wall and the second side-wall ranges from about 5 degree to about 160 degrees.

B24. The bioreactor of any one of embodiments B15 to B23, wherein the angle between the first side-wall and the second side-wall ranges from about 30 degrees to about 120 degrees.

B25. The bioreactor of any one of embodiments B15 to B24, wherein the angle between the first side-wall and the second side-wall is designed to optimize gas exchange between a fluid contained within the toroidal container and an overlaying gas contained within the toroidal container.

B26. The bioreactor of any one of embodiments B15 to B25, wherein the angle between the first side-wall and the second side-wall is designed to maximize a surface area-to-fluid volume ratio for the toroidal container.

B27. The bioreactor of any one of embodiments B16 to B26, wherein the at least one of the first side-wall or the second-side wall that is curved has a shape configured to provide different interfacial surface area-to-fluid volume ratios for a gas-fluid interface within the toroidal container for different volumes of fluid contained within the toroidal container.

B28. The bioreactor of any one of embodiments B1 to B27, wherein a total fluid volume of the toroidal container is at least 2 milliliters, 4 milliliters, 6 milliliters, 8 milliliters, 10 milliliters, 20 milliliters, 30 milliliters, 40 milliliters, 50 milliliters, 60 milliliters, 70 milliliters, 80 milliliters, 90 milliliters, 0.1 liters, 0.2 liters, 0.3 liters, 0.4 liters, 0.5 liters, 0.6 liters, 0.7 liters, 0.8 liters, 0.9 liters, 1 liters, 1.2 liters, 1.4 liters, 1.6 liters, 1.8 liters, or 2 liters.

B29. The bioreactor of any one of embodiments B1 to B28, wherein the at least one fluid inlet provides fluid communication between the stationary external fluid source and the interior of the toroidal container through a rotating fluid connector.

B30. The bioreactor of any one of embodiments B1 to B29, wherein the at least one fluid outlet provides fluid communication between the interior of the toroidal container and the stationary exterior fluid reservoir through a rotating fluid connector.

B31. The bioreactor of any one of embodiments B1 to B30, wherein the at least one fluid outlet comprises a filtration membrane and is configured for periodic or continuous removal of a fluid contained within the toroidal container.

B32. A system comprising:
at least one toroidal container comprising:
a cross-sectional profile comprising a top plate, a first side-wall, and a second side-wall, and
at least one fluid inlet configured to provide fluid communication between a stationary external fluid source and an interior of the toroidal container as the toroidal bioreactor rotates about its axis; and
at least one fluid outlet configured to provide fluid communication between the interior of the toroidal container and a stationary exterior fluid reservoir as the toroidal bioreactor rotates about its axis; and
at least one drive mechanism comprising:
a frame for mounting the toroidal container, wherein the frame is configured to allow the toroidal container to spin around its rotational axis; and
at least one drive motor;
wherein the drive mechanism is configured to control the angular velocity of the toroidal container as a function of time.

B33. The system of embodiment B32, wherein the at least one toroidal container comprises at least one micro cavity feature integrated with the first side-wall and configured to sample an aliquot of fluid contained within the toroidal container.

B34. The system of embodiment B33, wherein the at least one micro cavity feature is integrated with a top edge of the first side-wall.

B35. The system of embodiment B32 or embodiment B33, wherein the at least one micro cavity feature is configured to sample an aliquot of fluid contained within the toroidal container when an actuation force is applied to the at least one micro cavity feature.

B36. The system of any one of embodiments B33 to B35, wherein the at least one micro cavity feature is configured to deliver the fluid aliquot to a stationary external fluid sample container.

B37. The system of any one of embodiments B33 to B36, wherein the at least one micro cavity feature comprises:
a sample fluid inlet configured to provide fluid communication between an interior volume of the micro cavity feature and a fluid contained within the toroidal container;
a valve mechanism integrated with the sample fluid inlet and configured to open when an actuation force is applied;
the interior volume of the micro cavity feature;
a return fluid outlet configured to vent air and/or excess fluid from the interior volume of the micro cavity feature; and
a sample fluid outlet configured to provide fluid communication between the interior volume of the micro cavity feature and a stationary external fluid sample container.

B38. The system of embodiment B37, wherein the valve mechanism of the at least one micro cavity feature comprises a moveable component configured to toggle the valve between:
(i) an open sample fluid inlet/closed sample fluid outlet configuration, and
(ii) a closed sample fluid inlet/open sample fluid outlet configuration,
upon application of the actuation force.

B39. The system of any one of embodiments B35 to B38, wherein the actuation force comprises a centrifugal force applied when an angular velocity for rotation of the toroidal container about its axis exceeds a specified minimum angular velocity threshold.

B40. The system of any one of embodiments B35 to B39, wherein the actuation force comprises a magnetic force.

B41. The system of any one of embodiments B38 to B40, wherein the moveable component comprises a rolling element that rolls between a first indent and a second indent, wherein the first indent and second indent correspond to the open sample fluid inlet/closed sample fluid outlet configuration and the closed sample fluid inlet/open sample fluid outlet configuration, respectively.

B42. The system of any one of embodiments B37 to B41, wherein the interior volume of the at least one micro cavity feature at least 5 microliters, 10 microliters, 20 microliters, 30 microliters, 40 microliters, 50 microliters, 60 microliters, 70 microliters, 80 microliters, 90 microliters, 0.1 milliliters, 0.2 milliliters, 0.3 milliliters, 0.4 milliliters, 0.5 milliliters, 0.6 milliliters, 0.7 milliliters, 0.8 milliliters, 0.9 milliliters, 1 milliliters, 2 milliliters, 3 milliliters, 4 milliliters, or 5 milliliters.

B43. The system of any one of embodiments B32 to B42, wherein the frame comprises a self-centering engagement mechanism for attachment to a robotic actuator.

B44. The system of any one of embodiments B32 to B43, wherein the at least one drive motor of the drive mechanism comprises a servo motor.

B45. The system of any one of embodiments B32 to B44, wherein the at least one drive mechanism further comprises a self-centering engagement mechanism for attachment to a robotic actuator.

B46. The system of any one of embodiments B32 to B45, wherein the at least one drive mechanism further comprises a self-centering engagement mechanism for attachment to a rack configured to hold a plurality of drive mechanisms.

B47. The system of any one of embodiments B32 to B46, wherein the at least one drive mechanism further comprises at least one optical encoder for monitoring the angular velocity of the toroidal container as a function of time.

B48. The system of any one of embodiments B32 to B47, wherein the at least one drive mechanism is configured to vary the angular velocity of the toroidal container as a function of time in a programmable fashion.

B49. The system of any one of embodiments B33 to B48, wherein a variation in the angular velocity of the toroidal container as a function of time is used to control a composition of a fluid mixture that enters the at least one micro cavity feature.

B50. The system of any one of embodiments B32 to B49, wherein the at least one drive mechanism, or a housing thereof, further comprises at least one optical imaging camera configured to perform high speed imaging of particles suspended in the fluid contained within the toroidal container.

B51. The system of any one of embodiments B32 to B50, wherein the at least one drive mechanism, or a housing thereof, further comprises at least one optical imaging camera configured to perform high speed imaging of particles attached to an interior surface of the first side-wall of the toroidal container.

B52. The system of any one of embodiments B32 to B51, wherein the at least one drive mechanism, or a housing thereof, further comprises at least one optical sensor configured to perform optical sensing of at least one of pH, dissolved oxygen (DO), and temperature in a fluid contained within the toroidal container.

B53. The system of any one of embodiments B32 to B52, wherein the system further comprises a gas controller configured to provide control of the flow rate for at least one gas flowing into the toroidal container.

B54. The system of any one of embodiments B32 to B53, wherein the system further comprises a housing configured to provide temperature control for the toroidal container.

B55. The system of any one of embodiments B33 to B54, wherein a geometry of the at least one microcavity feature is configured to modify a shape of a cell that enters therein.

B56. The system of any one of embodiments B33 to B55, wherein a geometry of the at least one microcavity feature is configured to lyse a cell that enters therein.

B57. The system of any one of embodiments B33 to B56, further comprising an electromagnetic device configured to modify a cell that enters the at least one microcavity feature.

B58. The system of any one of embodiments B33 to B57, further comprising an electronic device configured to electroporate a cell that enters the at least one microcavity feature.

B59. A method for culturing cells, the method comprising:
introducing cells into a fluid contained within a bioreactor comprising:
a toroidal container having a cross-sectional profile comprising a top plate, a first side-wall, and a second side-wall, and
optionally, at least one micro cavity feature integrated with the first side-wall and configured to perform at least one of:
(i) sample an aliquot of fluid contained within the toroidal container when a force is applied to a valve mechanism integrated with the at least one micro cavity feature; and/or
(ii) deliver the fluid aliquot to a stationary external fluid sample container;
aerating and/or agitating the fluid contained within the toroidal container by rotating the toroidal container about its axis at a specified angular velocity; and
monitoring cell growth within the fluid by at least one of:
performing high speed imaging of cells suspended in the fluid,
performing high speed imaging of cells attached to an interior surface of the first side wall of the toroidal container, or
performing high speed imaging of cells contained in the at least on micro-cavity feature; and
sampling an aliquot of the fluid using the at least one micro cavity feature by increasing an angular velocity for rotation of the toroidal container so that it exceeds a specified minimum angular velocity threshold, and
delivering the aliquot of the growth medium to a stationary external sample container for testing.

B60. The method of embodiment B59, further comprising use of different angular velocity settings to perform different cell culture functions.

B61. The method of embodiment B60, wherein the different cell culture functions comprise mixing, resuspension, temperature control, cluster prevention, delivery of an aliquot of fluid to the at least one micro-cavity for cell analysis and/or modification, or any combination thereof.

B62. The method of any one of embodiments B59 to B61, further comprising monitoring at least one of pH, dissolved oxygen (DO), and temperature within the growth medium as a function of time.

B63. The method of any one of embodiments B59 to B62, wherein, prior to the sampling step, an angular velocity for rotation of the toroidal container is adjusted to select a subset of cells for sampling by the at least one micro cavity feature.

B64. The method of any one of embodiments B59 to B63, wherein, prior to the sampling step, an external force is applied to select a subset of cells for sampling by the at least one micro cavity feature.

B65. The method of embodiment B64, wherein the cells are attached to magnetic beads and the external force is a magnetic force.

### EXAMPLES

The presently disclosed subject matter will be better understood by reference to the following Examples, which are provided as exemplary of the invention, and not by way of limitation.

### Example 1—Toroidal Bioreactor Container With S-Shaped Side-Wall

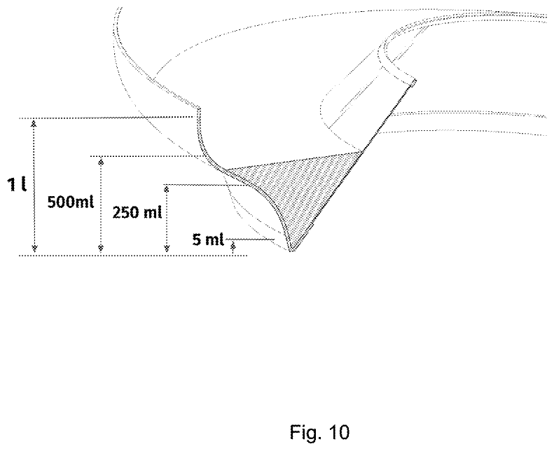

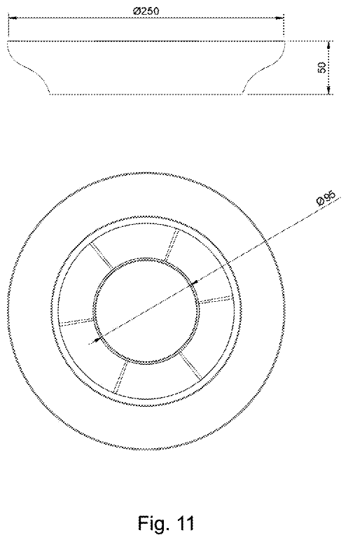

The example illustrated in FIG. 10 and FIG. 11 shows an embodiment of a bioreactor comprising an outer side-wall with an S-shaped curvature. This configuration of the disclosed bioreactor containers allows one to grow cells in the same container as the fluid volume varies from <5 ml to about 1000 ml. As shown in FIG. 10, a container with an S-shaped outer wall and the radial dimensions illustrated in FIG. 11 (i.e., an outer diameter of 250 mm, and inner diameter of 95 mm, and an overall height of 50 mm), allows one to culture cells (or other bioparticles) in different total volumes.

What is claimed is:

1. A bioreactor comprising:
a toroidal container having a cross-sectional profile comprising a top plate, a first side-wall, and a second side-wall;
at least one fluid inlet configured to provide fluid communication between a stationary external fluid source and an interior of the toroidal container as the toroidal bioreactor rotates about its axis; and
at least one fluid outlet configured to provide fluid communication between the interior of the toroidal container and a stationary exterior fluid reservoir as the toroidal bioreactor rotates about its axis.

2. The bioreactor of claim 1, further comprising at least one micro cavity feature integrated with the first side-wall and configured to remove an aliquot of fluid contained within the toroidal container.

3. The bioreactor of claim 2, wherein the at least one micro cavity feature is integrated with a top edge of the first side-wall.

4. The bioreactor of claim 2, wherein the at least one microcavity feature is configured to remove an aliquot of fluid contained within the toroidal container when an actuation force is applied to the at least one micro cavity feature.

5. The bioreactor of claim 2, wherein the at least one micro cavity feature is configured to deliver the fluid aliquot to a stationary external fluid sample container.

6. The bioreactor of claim 2, wherein the at least one micro cavity feature comprises:
a fluid inlet configured to provide fluid communication between an interior volume of the micro cavity feature and a fluid contained within the toroidal container;
a valve mechanism integrated with the sample fluid inlet and configured to open when an actuation force is applied;
the interior volume of the micro cavity feature;
a return fluid outlet configured to vent air and/or excess fluid from the interior volume of the micro cavity feature; and
a sample fluid outlet configured to provide fluid communication between the interior volume of the micro cavity feature and a stationary external fluid sample container.

7. The bioreactor of claim 6, wherein the valve mechanism of the at least one micro cavity feature comprises a moveable component configured to toggle the valve between:
(i) an open sample fluid inlet/closed sample fluid outlet configuration, and
(ii) a closed sample fluid inlet/open sample fluid outlet configuration,
upon application of the actuation force.

8. The bioreactor of claim 4, wherein the actuation force comprises a centrifugal force applied when an angular velocity for rotation of the toroidal container about its axis exceeds a specified minimum angular velocity threshold.

9. The bioreactor of claim 4, wherein the actuation force comprises a magnetic force.

10. The bioreactor of claim 8, wherein the moveable component comprises a movable element that moves between a first indent and a second indent, wherein the first indent and second indent correspond to the open sample fluid inlet/closed sample fluid outlet configuration and the closed sample fluid inlet/open sample fluid outlet configuration, respectively.

11. The bioreactor of claim 1, wherein the first side-wall and the second side-wall are slanted so as to converge at a third imaginary surface that defines an angle between the first side-wall and the second side-wall.

12. The bioreactor of claim 1, further comprising a bottom plate that intersects with the first side-wall and the second side-wall and is parallel to the top plate.

13. The bioreactor of claim 1, wherein at least a portion of the top plate, the first side-wall, and/or the second side-wall is composed of a gas permeable membrane.

14. The bioreactor of claim 1, wherein the first side-wall is an exterior side-wall, and wherein at least a portion of the first side-wall is optically transparent and configured to enable high speed imaging of the fluid contained within the toroidal container as the toroidal container rotates about its axis.

15. The bioreactor of claim 1, wherein the first side-wall is an exterior side-wall, and wherein at least a portion of the first side-wall is optically transparent and configured to enable optical sensing of at least one of pH, dissolved oxygen (DO), O2, N2, CO2, metabolomic profile, glutamate, glutamine, lactate, glucose, volume, flow rate, pressure, and temperature in a fluid contained within the toroidal container as the toroidal container rotates about its axis.

16. The bioreactor of claim 14, wherein the fluid contains particles such as cells, virus particles, beads, or any combination thereof.

17. The bioreactor of claim 11, wherein the at least one of the first side-wall or the second-side wall that is curved has a shape configured to provide different interfacial surface area-to-fluid volume ratios for a gas-fluid interface within the toroidal container for different volumes of fluid contained within the toroidal container.

18. The bioreactor of claim 1, wherein a total fluid volume of the toroidal container is at least 2 milliliters, 4 milliliters, 6 milliliters, 8 milliliters, 10 milliliters, 20 milliliters, 30 milliliters, 40 milliliters, 50 milliliters, 60 milliliters, 70 milliliters, 80 milliliters, 90 milliliters, 0.1 liters, 0.2 liters, 0.3 liters, 0.4 liters, 0.5 liters, 0.6 liters, 0.7 liters, 0.8 liters, 0.9 liters, 1 liters, 1.2 liters, 1.4 liters, 1.6 liters, 1.8 liters, or 2 liters.

19. The bioreactor of claim 1, wherein the at least one fluid outlet comprises a filtration membrane and is configured for periodic or continuous removal of a fluid contained within the toroidal container.

20. The bioreactor of claim 1, wherein the cross-sectional profile of the toroidal container is variable among sections of the circumference.

21. A system comprising:
at least one toroidal container comprising:
a cross-sectional profile comprising a top plate, a first side-wall, and a second side-wall, and
at least one fluid inlet configured to provide fluid communication between a stationary external fluid source and an interior of the toroidal container as the toroidal bioreactor rotates about its axis; and
at least one fluid outlet configured to provide fluid communication between the interior of the toroidal container and a stationary exterior fluid reservoir as the toroidal bioreactor rotates about its axis; and
at least one drive mechanism comprising:
a frame for mounting the toroidal container, wherein the frame is configured to allow the toroidal container to spin around a rotational axis; and
at least one drive motor;
wherein the drive mechanism is configured to control the angular velocity of the toroidal container as a function of time.

22. The system of claim 21, wherein the at least one toroidal container comprises at least one micro cavity feature integrated with the first side-wall and configured to remove an aliquot of fluid contained within the toroidal container when the container is rotated with a specified rotation sequence.

23. The system of claim 22, wherein the at least one micro cavity feature is integrated with a top edge of the first side-wall.

24. The system of claim 21, wherein the at least one micro cavity feature is configured to remove an aliquot of fluid contained within the toroidal container when an actuation force is applied to the at least one micro cavity feature.

25. The system of claim 22, wherein the at least one micro cavity feature is configured to deliver the fluid aliquot to a stationary external fluid sample container.

26. The system of claim 21, wherein the cross-sectional profile of the toroidal container is variable among sections of the circumference.

27. The system of claim 22, wherein the at least one micro cavity feature comprises:
a sample fluid inlet configured to provide fluid communication between an interior volume of the micro cavity feature and a fluid contained within the toroidal container;
a valve mechanism integrated with the sample fluid inlet and configured to open when an actuation force is applied;
the interior volume of the micro cavity feature;
a return fluid outlet configured to vent air and/or excess fluid from the interior volume of the micro cavity feature; and
a sample fluid outlet configured to provide fluid communication between the interior volume of the micro cavity feature and a stationary external fluid sample container.

28. The system of claim 27, wherein the valve mechanism of the at least one micro cavity feature comprises a moveable component configured to toggle the valve between:
(i) an open sample fluid inlet/closed sample fluid outlet configuration, and
(ii) a closed sample fluid inlet/open sample fluid outlet configuration,
upon application of the actuation force.

29. The system of claim 24, wherein the actuation force comprises a centrifugal force applied when an angular velocity for rotation of the toroidal container about its axis exceeds a specified minimum angular velocity threshold.

30. The system of claim 24, wherein the actuation force comprises a magnetic force.

31. The system of claim 28, wherein the moveable component comprises a movable element that moves between a first indent and a second indent, wherein the first indent and second indent correspond to the open sample fluid inlet/closed sample fluid outlet configuration and the closed sample fluid inlet/open sample fluid outlet configuration, respectively.

32. The system of claim 27, wherein the interior volume of the at least one micro cavity feature at least 5 microliters, 10 microliters, 20 microliters, 30 microliters, 40 microliters, 50 microliters, 60 microliters, 70 microliters, 80 microliters, 90 microliters, 0.1 milliliters, 0.2 milliliters, 0.3 milliliters, 0.4 milliliters, 0.5 milliliters, 0.6 milliliters, 0.7 milliliters, 0.8 milliliters, 0.9 milliliters, 1 milliliters, 2 milliliters, 3 milliliters, 4 milliliters, or 5 milliliters.

33. The system of claim 21, wherein the at least one drive mechanism further comprises a self-centering engagement mechanism for attachment to a robotic actuator.

34. The system of claim 21, wherein the at least one drive mechanism further comprises a self-centering engagement mechanism for attachment to a rack configured to hold a plurality of drive mechanisms.

35. The system of claim 21, wherein the at least one drive mechanism is configured to vary the angular velocity of the toroidal container as a function of time in a programmable fashion.

36. The system of claim 22, wherein a variation in the angular velocity of the toroidal container as a function of time is used to control a composition of a fluid mixture that enters the at least one micro cavity feature.

37. The system of claim 21, wherein the at least one drive mechanism, or a housing thereof, further comprises at least one optical imaging camera configured to perform high speed imaging of the fluid contained within the toroidal container.

38. The system of claim 21, wherein the at least one drive mechanism, or a housing thereof, further comprises at least one optical sensor configured to perform optical sensing of at least one of pH, dissolved oxygen (DO), and temperature in a fluid contained within the toroidal container.

39. The system of claim 21, wherein the system further comprises a gas controller configured to provide control of the flow rate for at least one gas flowing into the toroidal container.

40. The system of claim 21, wherein the system further comprises a housing configured to provide temperature control for the toroidal container.

41. The system of claim 22, wherein a geometry of the at least one microcavity feature is configured to modify a shape of a cell that enters therein.

42. The system of claim 22, wherein a geometry of the at least one microcavity feature is configured to lyse a cell that enters therein.

43. The system of claim 22, further comprising an electromagnetic device configured to modify a cell that enters the at least one microcavity feature.

44. The system of claim 22, further comprising an electronic device configured to electroporate a cell that enters the at least one microcavity feature.

45. A method for culturing cells, the method comprising:
introducing cells into a fluid contained within a bioreactor comprising:
a toroidal container having a cross-sectional profile comprising a top plate, a first side-wall, and a second side-wall, and
optionally, at least one micro cavity feature integrated with the first side-wall and configured to perform at least one of:
(i) remove an aliquot of fluid contained within the toroidal container when a force is applied to a valve mechanism integrated with the at least one micro cavity feature; and/or
(ii) deliver the fluid aliquot to a stationary external fluid sample container;
aerating and/or agitating the fluid contained within the toroidal container by rotating the toroidal container about its axis at a specified angular velocity; and
monitoring cell growth within the fluid by at least one of:
performing high speed imaging of cells suspended in the fluid,
performing high speed imaging of cells attached to an interior surface of the first side wall of the toroidal container, or
performing high speed imaging of cells contained in the at least on micro-cavity feature; and
sampling an aliquot of the fluid using the at least one micro cavity feature by increasing an angular velocity for rotation of the toroidal container so that it exceeds a specified minimum angular velocity threshold, and
delivering the aliquot of the growth medium to a stationary external sample container for testing.

46. The method of claim 45, further comprising use of different angular velocity settings to perform different cell culture functions.

47. The method of claim 46, wherein the different cell culture functions comprise mixing, resuspension, temperature control, cluster prevention, delivery of an aliquot of fluid to the at least one micro-cavity for cell analysis and/or modification, or any combination thereof.

48. The method of claim 45, further comprising monitoring at least one of pH, dissolved oxygen (DO), and temperature within the growth medium as a function of time.

49. The method of claim 45, wherein, prior to the sampling step, an angular velocity for rotation of the toroidal container is adjusted to select a subset of cells for sampling by the at least one micro cavity feature.

50. The method of claim 45, wherein, prior to the sampling step, an external force is applied to select a subset of cells for sampling by the at least one micro cavity feature.

51. The method of claim 50, wherein the cells are attached to magnetic beads and the external force is a magnetic force.

52. The method of claim 45, wherein the cross-sectional profile of the toroidal container is adjustable such that one or more sections of the toroidal container vary relative to one another.
